(19) United States
(12) Patent Application Publication
(10) Pub. No.: US 2015/0352161 A1
(43) Pub. Date: Dec. 10, 2015

(54) USE OF HAFNIA ALVEI FOR REDUCING THE CARRIAGE OF ESCHERICHIA COLI PRODUCING SHIGA TOXINS (STEC) IN RUMINANTS

(71) Applicants: INSTITUT NATIONAL DE LA RECHERCHE AGRONOMIQUE, Paris (FR); DANSTAR FERMENT AG, Zug (CH)

(72) Inventors: Evelyne FORANO, La Roche Blanche (FR); Yolande BERTIN, Romagnat (FR); Marie-Christine MONTEL, Aurillac (FR); Frederique CHAUCHEYRAS-DURAND, Aurieres (FR)

(21) Appl. No.: 14/763,615

(22) PCT Filed: Jan. 28, 2014
(86) PCT No.: PCT/EP2014/051563

§ 371 Date: Jul. 27, 2015

(30) Foreign Application Priority Data
Jan. 28, 2013 (FR) 1350697

(51) Int. Cl. A61K 35/74; A23K 1/18; A23K 1/00; A61K 36/064

(52) U.S. Cl. CPC A61K 35/74; A61K 36/064; A23K 1/1813; A23K 1/009

(57) ABSTRACT

The invention relates to a method for reducing the risk of contamination of milk and meat when milking and slaughtering ruminants carrying STEC. The invention especially relates to a method for reducing the carriage of STEC by ruminants, characterised by the administration of at least one strain of Hafnia alvei to the ruminant. The invention also relates to a composition comprising at least one strain of Hafnia alvei combined with a strain of Saccharomyces boulardii.

### FIELD OF THE INVENTION

The present invention relates to the use of a strain of Hafnia alvei as a feed additive in animal nutrition for reducing the carriage of Shiga toxin-producing Escherichia coli in ruminants.

### STATE OF THE ART

Shiga toxin-producing Escherichia coli (STEC), also called enterohemorrhagic Escherichia coli (EHEC), and more specifically serotype O157:H7, are currently recognized as emergent pathogens important for public health. These bacteria have been detected in a large number of animal species not exhibiting clinical signs of disease. The principal “reservoirs” or “carriers” are ruminants, more particularly cattle. The prevalence of STEC in cattle feces varies from 4% to 36%. The persistence of STEC strains in livestock is due in part to “intestinal carriage” by these animals. Carriage varies by season, with prevalence being higher during spring and summer.

The risk of contamination of foodstuffs of animal origin intended for human consumption is related to the hygiene procedures in slaughterhouses and meat processing units, but also to the level of animal carriage. When ingested by humans, these bacteria most commonly cause hemorrhagic colitis. Hemolytic-uremic syndrome may also appear in children and in the elderly, as well as thrombotic thrombocytopenic purpura in adults.

To reduce STEC carriage in ruminants, it has been suggested to administer as a feed additive to said ruminants bacteria such as Lactobacillus acidophilus strains which reduce the prevalence of E. coli O157 in feces (Stephens et al., 2007; Tabe et al., 2008). It has also been shown in vitro that a Lactobacillus acidophilus strain and a Saccharomyces cerevisiae strain reduce STEC survival time in fluid of the paunch (rumen), the first compartment of the complex digestive system of ruminants (Chaucheyras-Durand et al., 2010).

Hafnia alvei is an enterobacterium found in human and animal gut flora. This bacterium is used in the cheese industry to reproduce the taste of rawmilk cheeses in cheeses prepared from pasteurized milk (Delbès-Paus et al., 2012) or to reduce the growth of E. coli O26:H11 in cheese (Delbès-Paus et al., 2013). Hafnia alvei is used in particular in a composition for flavoring cheeses, also comprising Lactobacillus and at least one yeast (patent application WO 99/43214). Hafnia alvei can also be used to preserve food products, via inoculation, in order to competitively inhibit the development of pathogenic bacteria (U.S. Pat. No. 5,869,113).

However, the properties of Hafnia alvei in foodstuffs of animal origin should be distinguished from its desired effect in live animals. In particular, an antagonistic effect demonstrated on the surface of meat or in a cheese matrix is not predictive of an inhibitory effect on STEC growth-survival under conditions of the animal digestive tract or of a representative in vitro model system. Indeed, the physicochemical conditions of a cheese matrix and of animal digestive contents are very different (a much lower redox potential in the digestive tract which is an essentially anaerobic medium; a more acidic pH in cheeses; a lower water activity (aw) in cheese due to the addition of salt; a variety of substrates available in milk—namely lactose and other sugars, proteins and fats—whereas in the digestive tract of ruminants they are essentially plant polysaccharides). The microorganisms present therein are also very different with regard to which microbial species are present and to the diversity and population levels thereof; lactic acid bacteria are predominant in raw milk and in cheeses (Masoude et al., 2012), whereas in the digestive ecosystems of ruminants the principal bacterial genera are Prevotella, Ruminococcus, Butyrivibrio, Clostridium, etc. (Jami & Mizrahi, 2012; de Olivera et al., 2013).

Thus, the inventors have shown that the following microorganisms, for example, have an inhibitory effect on STEC growth (at least equal to 1 log: difference in growth of an EHEC strain with or without the inhibitory strain) in a cheese matrix (uncooked pressed cheese), and in particular with strains O157:H7 or O26:H11, whereas they do not have an inhibitory effect on these strains incubated in animal digestive contents in vitro: Leuconostoc pseudomesenteroides, strain MSE7; Leuconostoc citreum, strain MSE2; Leuconostoc citreum, strain MSE30.5; Marinilactibacillus psychotolerans, strain CRBM29; Carnobacterium mobile, strain CRBM18; Enterococcus faecalis, strain N688; Macrococcus caseolyticus, strain RPB; Lactococcus lactis subsp. lactis, strain 1147; Lactococcus lactis subsp. lactis, strain 278m; Lactococcus lactis subsp. cremoris, strain N718; Lactococcus lactis, strain D5.3; Lactobacillus plantarum, strain FH3.

### SUMMARY OF THE INVENTION

To date there is no method available to industrial producers for limiting the carriage of STEC in ruminants. The only way to limit the contamination of milk or meat is to ensure excellent hygiene when the animals are milked or slaughtered. Reducing carriage would reduce the risk of direct contamination of humans via contact with the animals, via products derived therefrom or via the consumption of contaminated plants.

The present application relates to a method for reducing the risk of contamination of ruminant milk or meat when animals carrying STEC are milked or slaughtered. The risk of contamination is reduced in particular by reducing the carriage of STEC by said ruminants.

The present invention relates in particular to a method for reducing the carriage of STEC in ruminants, characterized by the administration to a ruminant of one or more strains of the species Hafnia alvei. Said administration is easily accomplished orally by means of a feed additive.

### DETAILED DESCRIPTION OF THE INVENTION

The invention relates to a method for reducing the carriage of STEC in ruminants, in particular serotypes O157:H7 and O26:H11, characterized by the administration of at least one strain of Hafnia alvei to a ruminant.

The term “carriage” or “animal carriage” refers to the fact that STEC bacteria naturally reside in the body of the animals that “carry” them, without causing harm and, in particular, with no clinical sign of disease being detectable, although the animals' feces contain said STEC. Although the animals carry STEC bacteria, the present invention does not relate to a method for treating the animals but to a method for reducing the amount of STEC present in the intestinal pouches of said animals and thus for reducing the risk of contamination of the milk during milking and of the meat during slaughter of said animals, or of the feces of said animals.

The term “STEC” refers to Shiga toxin-producing Escherichia colt bacteria; this group (as defined in the 2003 AFSSA report) comprises in particular serotypes O157:H7 and O26:H11.

The term “ruminants” refers to herbivorous animals able to regurgitate food in order to chew it again, in other words to ruminate. By “ruminants” is meant essentially animals of the suborder Ruminantia, in particular cattle, sheep and goats. The present invention relates in particular to commercial cattle.

The term “administration” encompasses oral, intravenous or intramuscular administration. Nevertheless, the present invention essentially relates to oral administration.

The term “strain(s) of Hafnia alvei” refers to any strain or combination of strains related to this Hafnia subfamily, known since the 1960s. Hafnia strains have been isolated in particular from raw milk and from cheese. There are two large families, Hafnia alvei and Hafnia paralvei. Certain strains can be virulent (Abbott et al., 2011). Naturally, the present invention relates to the use of (a) nonvirulent, nonpathogenic strain(s) of Hafnia alvei.

According to a preferred aspect of the invention, the strain(s) is (are) included in a feed additive intended for ruminants. A “feed additive” is distinguished from a feedstuff by the fact that a small amount is used in animal feedstuffs. In Europe, feed additives for livestock development are covered by Regulation (EC) No 1831/2003 of the European Parliament and of the Council. Industrial producers may, under certain conditions, add to animal feedstuffs:
“technological additives”; preservatives, antioxidants, emulsifiers, stabilizers, thickeners, gelling agents, binders, substances for control of radionucleide contamination, anticaking agents, acidity regulators, silage additives, denaturants;
“sensory additives”; colorants (substances that add or restore color in feedstuffs; substances which, when fed to animals, add colors to food produced from said animals. Certain pigments which favorably affect the color of ornamental fish or birds are also authorized);
“flavoring substances”;
“nutritional additives,” including vitamins, provitamins and “chemically well-defined substances having similar effect,” compounds of trace elements, amino acids (and their salts and “analogues,” and urea and its derivatives;
“zootechnical additives,” authorized as digestibility enhancers, gut flora stabilisers, substances “which favorably affect the environment,” and “other zootechnical additives”.

Preferentially, the feed additive comprising the Hafnia alvei strain(s) will be mixed with a feedstuff intended for ruminants. Preferably, the Hafnia alvei strain(s) is (are) in freeze-dried form, and in particular in powder form, in order to be easily added to said feedstuff.

According to a particular aspect of the invention, said feedstuff or feedstuff composition or feed additive comprises at least about 10⁸ CFU of Hafnia alvei per animal per day, advantageously about 10⁸ CFU to about 10¹² CFU of Hafnia alvei per animal per day. In cattle the preferred amount is about 10¹⁰ CFU per animal per day and in sheep and goats the preferred amount is about 10⁹ CFU per animal per day. The term “CFU” means “colony forming unit” and is the unit of measure generally recognized by the person skilled in the art for quantifying bacteria able to give rise to colonies.

In particular, the feedstuff composition may include other feed additives selected from the additives cited above and/or the following active ingredients: hormones and/or bactericides and/or antiinflammatories and/or antifungals.

According to an aspect of the invention, the strain of Hafnia alvei administered to a ruminant is selected from the strains deposited with the DSMZ under accession numbers 30098, 30099 or 30163.

According to a preferred aspect of the invention, the strain of Hafnia alvei administered to a ruminant is strain B16 deposited with the CNCM under number CNCM I-4710 on 20 Dec. 2012. This strain, previously called “CC16,” was isolated in 2009 from a French “Livarot” cheese (Mounier et al., 2009).

According to a specific aspect of the invention, the strain of Hafnia alvei is administered in combination with at least one other microorganism strain, and in particular another strain of Hafnia alvei and/or a yeast. In the case of a combination with a yeast, the yeast is in particular the strain of Saccharomyces boulardii deposited with the CNCM under accession number I-1079.

According to another specific aspect of the invention, several strains of Hafnia alvei are combined in order to be administered to a ruminant in the context of the method according to the invention.

The invention also relates to a composition comprising a strain of Hafnia alvei in combination with at least one other microorganism strain. In particular, this other microorganism strain is another strain of Hafnia alvei and/or a yeast. In a preferred manner, this yeast is the specific species Saccharomyces boulardii. In particular, the strain of Saccharomyces boulardii present in the composition is the strain deposited with the CNCM under accession number I-1079.

In particular, the strain of Hafnia alvei present in said composition is strain B16 deposited with the CNCM under accession number I-4710, in combination with the strain of Saccharomyces boulardii deposited with the CNCM under accession number I-1079.

According to another embodiment of the invention, the composition comprises a strain of Hafnia alvei, in particular one of the strains deposited with the DSMZ under accession number 30098, 30099 or 30163, and the strain of Saccharomyces boulardii deposited with the CNCM under accession number I-1079.

According to another embodiment of the invention, the composition further comprises at least one other microorganism strain. This other microorganism can be in particular a strain of Hafnia alvei, in particular one of the strains deposited with the DSMZ under accession number 30098, 30099 or 30163, or any other suitable microorganism.

According to another embodiment, the composition comprises strain B16 deposited with the CNCM under accession number I-4710, at least one strain deposited with the DSMZ under accession number 30098, 30099 or 30163, and the strain of Saccharomyces boulardii deposited with the CNCM under accession number I-1079.

The invention also relates to the use of the one of the compositions as defined above as a feed additive for ruminants. Said composition will be preferentially added to a feedstuff for animals. Said composition will be used to reduce the carriage of STEC in ruminant animals fed this composition.

Lastly, the invention relates to the use of a strain of Hafnia alvei, alone or in combination with other microorganism strains, for reducing the carriage of STEC in ruminants.

STEC carriage in ruminants is evaluated by conventional methods well-known to the person skilled in the art. In particular, the level of carriage in vivo is evaluated by measuring the prevalence of STEC present in a herd and the level and/or duration of excretion of STEC in the animals' feces, as described in Tabe et al., 2008, for example. The method according to the invention makes it possible to obtain a reduction of fecal excreta of at least 10% compared with ruminants not having absorbed at least one strain of Hafnia alvei; preferentially, the reduction is at least 20%, 30%, 40%, 50%, 60%, 70%, 80%, 90%, 100%, 120%, 150%, 200% and even 300%. In a preferred manner, the prevalence of STEC and, in particular, serotypes O157:H7 and O26:H11 in the animals to which at least one strain of Hafnia alvei has been administered is reduced by about 30% compared with the prevalence of STEC in animals not having been treated according to the method of the invention. The inhibitory effect on STEC growth by a strain of Hafnia alvei can also be demonstrated in vitro by means of the growth of the strains in bovine ileal fluid, as presented in the examples below.

### DESCRIPTION OF THE FIGURES

FIG. 1. Growth of E. coli O157:H7 FCH6 in bovine ileal fluid alone (dark gray) or in the presence of a strain of Hafnia alvei (light gray)

FIG. 2. Growth of E. coli O26:H11 21765 in bovine ileal fluid alone (dark gray) or in the presence of a strain of Hafnia alvei (light gray)

FIG. 3. Growth of E. coli O157:H7 FCH6 and 21765 in bovine ileal fluid (dark gray) and in the presence of a mixture of Hafnia alvei B16 and the yeast S. boulardii I-1079 (light gray)

FIG. 4. Growth of E. coli O157:H7 FCH6 in bovine ileal fluid alone (dark gray) or in the presence of a mixture of two strains of Hafnia alvei, B16 and 30098 (clear gray)

### EXAMPLES

### Example 1

### Effect of Hafnia alvei B16 and Other Strains of Hafnia alvei on the Growth of E. coli O157:H7 FCH6 in Bovine Ileal Fluid

In vitro incubations were prepared in ileal fluid obtained from two cannulated cows receiving a ration (80:20, respectively) of natural meadow hay and of concentrate (INRA concentrate 863-50183 GV comprising beet pulp, wheat, barley, rapeseed cake, soybean cake, cane sugar molasses, calcium hydrogen phosphate, salt, magnesium oxide, vitamins, provitamins and trace elements). Tubes containing ileal fluid were incubated for 5 hours at 39° C., without shaking, in the presence of the strains of interest.

The potential inhibitory effect of each candidate strain was tested first on a rifampicin-resistant strain of O157:H7 (strain FCH6). The count of strain FCH6 before and after 5 hours of incubation was carried out on LB agar supplemented with rifampicin.

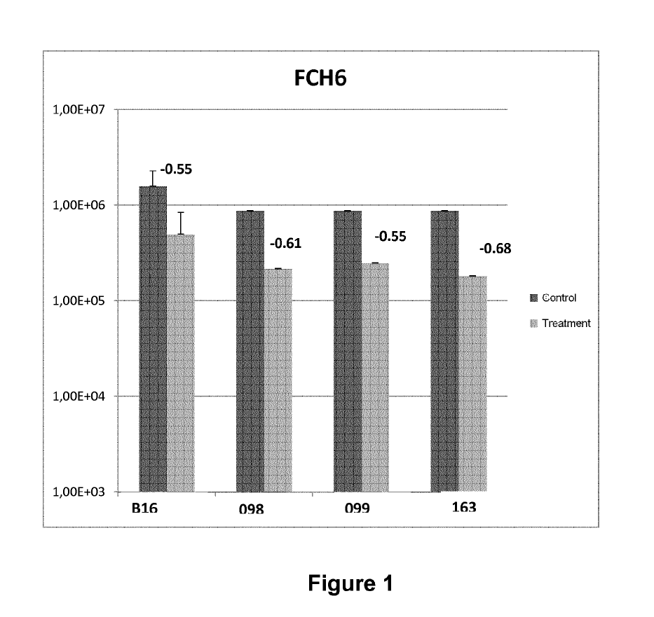

In our study, the addition of 500 μl of a 24-hour culture of Hafnia alvei B16 in BHI medium (not shaken) in 5 ml of ileal fluid inoculated with 10⁴ CFU/ml of Rif^{R} FCH6 significantly (p<0.001) and reproducibly reduced the growth of the STEC strain after 5 hours of incubation (−0.55 log of the mean population of 11 independent incubations; see FIG. 1). The other Hafnia alvei strains tested (DSMZ strains 30098, 30099 and 30163) also have an inhibitory effect.

The results presented in FIG. 1 show that Hafnia alvei strain B16 has a good ability to antagonize the growth of E. coli strain O157:H7.

### Example 2

### Effect of Hafnia alvei B16 and Other Hafnia alvei Strains on the Growth of E. coli O26:H11 21765 in Bovine Ileal Fluid

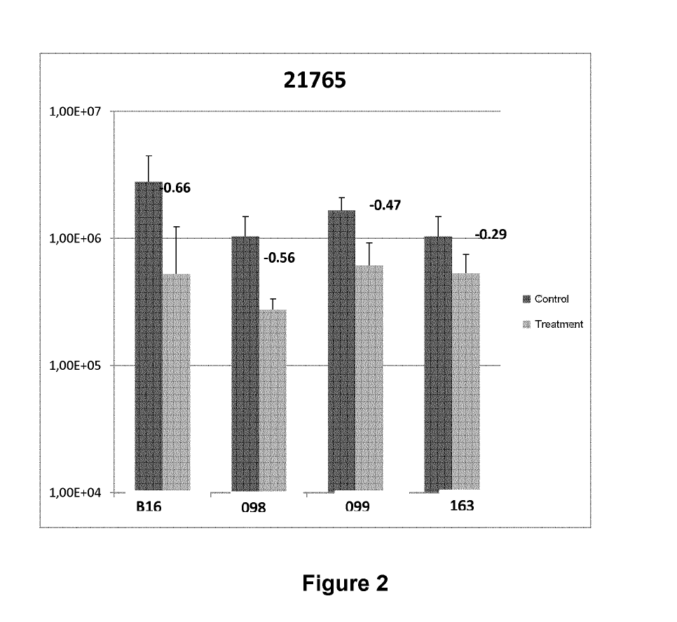

Comparable results were obtained with another STEC strain of serotype O26:H11 (strain Rif^{R} 21765). A significant (p<0.05) decrease in population growth of −0.66 log (mean of 9 independent incubations, FIG. 2) was indeed measured after 5 hours of incubation in bovine ileal fluid with strain B16. The other Hafnia alvei strains tested (DSMZ strains 30098, 30099 and 30163) also have an inhibitory effect on strain 21765.

### Example 3

### Effect of Hafnia alvei B16 in Combination with a Yeast Strain on the Growth of E. coli O157:H7 FCH6 and E. coli O26:H11 21765 in Bovine Ileal Fluid

The experimental conditions are identical to those described in Example 1, with the following exception: the inhibitory strains are added in the amount of 250 μl each and Saccharomyces boulardii is cultured beforehand in malt broth for 24 hours at 30° C. with gentle shaking.

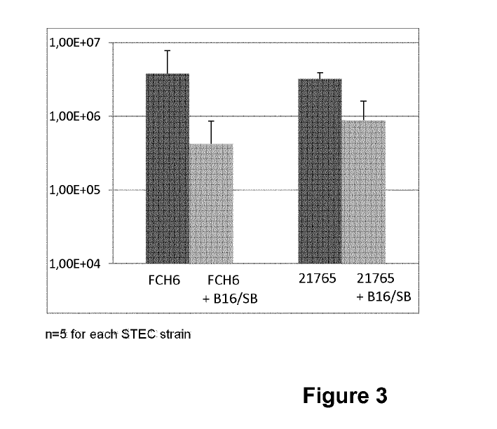

The results are presented in FIG. 3: for the strain FCH6, the reduction in growth is nearly 1 log (reduction by a factor of 10); for the strain 21765, the reduction in growth is −0.5 log.

### Example 4

### Effect of a Combination of Two Hafnia alvei Strains (B16 and 30098) on the Growth of E. coli O157:H7 FCH6 in Bovine Ileal Fluid

The experimental conditions are identical to those described in Example 1; the two strains are added in an amount of 250 μl each.

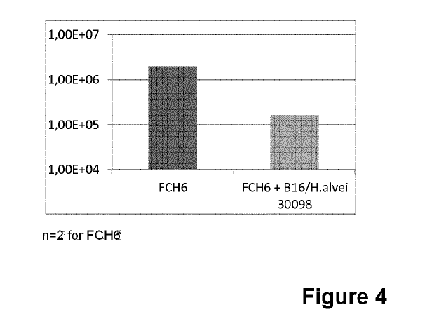

The results are presented in FIG. 4: for the strain FCH6, the reduction in growth is 1 log (reduction by a factor of 10) in the presence of the combination of the two Hafnia alvei strains.

### Example 5

### Verification of the Survival of the H. alvei Strain B16 in Ileal Contents In Vitro

Survival of the Hafnia alvei strain tested in Examples 1 to 4 above (B16) in ileal contents in vitro comprising endogenous gut flora was verified, in particular with 10⁸ CFU/ml of Hafnia alvei. The experimental conditions are identical to those described in Example 1, except for inoculation with the E. coli strains.

The results obtained show that the amount of Hafnia alvei B16 is maintained at the same level in intestinal fluid in vitro for more than 5 hours (results not shown).

### REFERENCES

### Patent Application/Patent

WO 99/43214

U.S. Pat. No. 5,869,113

### Bibliographical References in Alphabetical Order

AFSSA (2003) State of knowledge of Shiga toxin-producing Escherichia coli (STEC).
Chaucheyras-Durand F, Faqir F, Ameilbonne A, Rozand C, Martin C. Fates of acid-resistant and non-acid-resistant Shiga toxin-producing Escherichia coli strains in ruminant digestive contents in the absence and presence of probiotics. Appl Environ Microbiol. 2010 February;76(3):640-7.
Delbès-Paus C, Pochet S, Helinck S, Veisseire P, Bord C, Lebecque A, Coton M, Desmasures N, Coton E, Irlinger F, Montel M C. Impact of Gram-negative bacteria in interaction with a complex microbial consortium on biogenic amine content and sensory characteristics of an uncooked pressed cheese. Food Microbiol. 2012 May;30(1):74-82. Epub 2011 Dec. 14.
DelbèsPaus C, Miszczycha S, Ganet S, Helinck S, Veisseire P, Pochet S, Thèvenot D, Monter M C, Behavior of Escherichia coli O26:H11 in the presence of Hafnia alvei in a model cheese ecosystem, Int. J. Food Microbiol. 2013 160, 212-218.
de Oliveira M N, Jewell K A, Freitas F S, Benjamin L A, Tótola M R, Borges A C, Moraes C A, Suen G. Characterizing the microbiota across the gastrointestinal tract of a Brazilian Nelore steer. Vet Microbiol. 2013 Jun. 28;164(3-4):307-14.
Jami E, Mizrahi I. Composition and similarity of bovine rumen microbiota across individual animals. PLoS One. 2012;7(3):e33306.
Masoud W, Vogensen F K, Lillevang S, Abu Al-Soud W, Søorensen S J, Jakobsen M. The fate of indigenous microbiota, starter cultures, Escherichia coli, Listeria innocua and Staphylococcus aureus in Danish raw milk and cheeses determined by pyrosequencing and quantitative real time (qRT)-PCR. Int J Food Microbiol. 2012 Feb. 1;153(1-2):192-202.
Mounier J, Monnet C, Jacques N, Antoinette A, Irlinger F. Assessment of the microbial diversity at the surface of Livarot cheese using culture-dependent and independent approaches. Int J Food Microbiol. 2009 Jul. 31;133(1-2):31-7.
Stephens T P, Loneragan G H, Karunasena E, Brashears M M. Reduction of Escherichia coli O157 and Salmonella in feces and on hides of feedlot cattle using various doses of a direct-fed microbial. J Food Prot. 2007 October;70(10):2386-91.
Tabe E S, Oloya J, Doetkott D K, Bauer M L, Gibbs P S, Khaitsa M L. Comparative effect of direct-fed microbials on fecal shedding of Escherichia coli O157:H7 and Salmonella in naturally infected feedlot cattle. J Food Prot. 2008. 71(3):539-44. Erratum in: J Food Prot. 2008. 71(5):886.

1-13. (canceled)

14. A method for reducing the carriage of STEC in ruminants, comprising administering at least one strain of Hafnia alvei to a ruminant.

15. The method according to claim 14, wherein said strain is included in a feed additive intended for ruminants.

16. The method according to claim 15, wherein the at least one strain of Hafnia alvei is administered in an amount of at least about 10⁸ CFU per animal per day.

17. The method according to claim 15, wherein the at least one strain of Hafnia alvei is administered to cattle in an amount of about 10¹⁰ CFU per animal per day.

18. The method according to claim 15, wherein the at least one strain of Hafnia alvei is the strain B16 deposited with the CNCM under number I-4710.

19. The method according to claim 15, wherein the at least one strain of Hafnia alvei is administered in combination with at least one additional microorganism strain.

20. The method according to claim 19, wherein the at least one additional microorganism strain is a strain of Hafnia alvei, a strain of yeast, or both a strain of Hafnia alvei and a strain of yeast.

21. The method according to claim 20, wherein the strain of yeast is the strain of Saccharomyces boulardii deposited with the CNCM under accession number I-1079.

22. The method according to claim 18, wherein the at least one strain of Hafnia alvei is administered in combination with at least one additional microorganism strain.

23. The method according to claim 22, wherein the at least one additional microorganism strain is a strain of Hafnia alvei, a strain of yeast, or both a strain of Hafnia alvei and a strain of yeast.

24. The method according to claim 23, wherein the strain of yeast is the strain of Saccharomyces boulardii deposited with the CNCM under accession number I-1079.

25. A composition comprising an isolated strain of Hafnia alvei in combination with an isolated strain of Saccharomyces boulardii.

26. The composition according to claim 25, wherein the strain of Hafnia alvei is the strain B16 deposited with the CNCM under number I-4710 and the strain of Saccharomyces boulardii is the strain deposited with the CNCM under accession number I-1079.

27. The composition according to claim 25, further comprising at least one additional microorganism strain.

28. A feed additive for ruminants comprising the composition according to claim 25.

29. A feed additive for ruminants comprising the composition according to claim 26.

30. A feed additive for ruminants comprising the composition according to claim 27.
